(19) United States
(12) Patent Application Publication
(10) Pub. No.: US 2018/0256746 A1
(43) Pub. Date: Sep. 13, 2018

(54) IMMEDIATE RELEASE CLINDAMYCIN DELIVERY COMPOSITION AND FORMULATION

(71) Applicant: Iono Pharma, LLC, Bartlett, TN (US)

(72) Inventors: Hassan Almoazen, Bartlett, TN (US); Baher Daihom, El Rehab City (EG); Alaadin Alayoubi, Silver Springs, MD (US)

(73) Assignee: Iono Pharma, LLC, Bartlett, TN (US)

(21) Appl. No.: 15/911,079

(22) Filed: Mar. 3, 2018

Related U.S. Application Data
(60) Provisional application No. 62/469,052, filed on Mar. 9, 2017.

(51) Int. Cl. A61K 47/69; A61K 31/7056; A61K 9/00; A61P 31/04

(52) U.S. Cl. CPC A61K 47/6933; A61P 31/04; A61K 9/0053; A61K 31/7056

(57) ABSTRACT

This invention relates to a pharmaceutical composition made of a clindamycin salt complexed with strongly acidic non-spherical resin particles, wherein predominantly all of the resin particles are less than 75 microns in diameter, to form clindamycin-resin complex, wherein in the clindamycin-resin complex provides for immediate release of the clindamycin in a target environment and at least one non-ionic excipient. In one embodiment, the inventive subject matter is an oral liquid pharmaceutical formulation made of a complex of clindamycin hydrochloride, a fractionated resin and excipients to stabilize the complex. The pharmaceutical formulation remains stable in deionized water until the dosage form reaches an ion-rich environment (stomach or intestine), there which the active ingredient clindamycin hydrochloride is released.

### CROSS-REFERENCE TO RELATED APPLICATIONS

This application claims the benefit of U.S. Application Ser. No. 62/469,052 filed Mar. 9, 2017, under 35 U.S.C. 119(e), hereby specifically incorporated by reference in its entirety.

### STATEMENT REGARDING FEDERALLY SPONSORED RESEARCH OR DEVELOPMENT

None.

### THE NAMES OF THE PARTIES TO A JOINT RESEARCH AGREEMENT

None

### REFERENCE TO A “SEQUENCE LISTING”, A TABLE, OR A COMPUTER PROGRAM LISTING APPENDIX SUBMITTED ON A COMPACT DISC AND AN INCORPORATION-BY-REFERENCE OF THE MATERIAL ON THE COMPACT DISC

None.

### FIELD OF THE INVENTION

The inventive subject matter includes an oral liquid pharmaceutical formulation made of a clindamycin salt complexed with strongly acidic non-spherical resin particles, wherein predominantly all of the resin particles are less than 75 microns in diameter and at least one non-ionic excipient to form a clindamycin-resin complex, wherein in the clindamycin-resin complex provide for immediate release of the clindamycin in a target environment.

### BACKGROUND OF THE INVENTION

Clindamycin is an antimicrobial agent of the lincosamide class. Clindamycin has been widely used against a wide range of aerobic and anaerobic gram-positive bacteria involved in several inflammations as in acne vulgaris, toxoplasmosis, bacterial vaginosis and methicillin-resistant staphylococcus aureus MRSA. Sinkula A, Morozowich W, Rowere E. “Chemical Modification of Clindamycin: Synthesis and Evaluation of Selected Esters”, Journal of pharmaceutical sciences. 1973; 62(7):1106-11. However, the repulsive odor and taste of clindamycin presents a significant challenge for patient adherence to therapy. Mennella J A, Pepino M Y, Reed D R., “Genetic and environmental determinants of bitter perception and sweet preferences”, Pediatrics, 2005; 115(2):e216-e22.

Clindamycin is commercially available in a prodrug form (palmitate ester) at a concentration of 75 mg/5 ml. The palmitate ester was synthesized to reduce the bitterness of clindamycin. However, this chemical modification of clindamycin doesn't eliminate the bad taste completely. Therefore, a need exists to enhance clindamycin's taste and odor for better compliance. To date, several techniques have been used to mask the taste of pharmaceutical compounds including microencapsulation, and complexation. Tripathi A, Parmar D, Patel U, Patel G, Daslaniya D, Bhimani B. Taste Masking: “A Novel Approach for Bitter and Obnoxious Drugs”, JPSBR. 2011; 1(3):36-142.

Ion exchange resin (IER) complexation is a technique that has been used for sustained or controlled release drug delivery. Anand V, Kandarapu R, Garg S., “Ion-exchange resins: carrying drug delivery forward”, Drug Discovery Today. 2001; 6(17):905-14. Srikanth M, Sunil S, Rao N, Uhumwangho M, Murthy K R., “Ion-exchange resins as controlled drug delivery carriers”, Journal of Scientific Research. 2010; 2(3):597. The IER forms a complex that remains stable in deionized water until the dosage form reach an ion-rich environment (stomach or intestine) there which the drug gets released. This mechanism of binding minimizes any premature release of drug in the mouth during swallowing where the ions concentration is minimal and subsequently makes the drug palatable. However, for compounds with intense bitter taste such as clindamycin, any amount that is released from the resin complex due to the presence of some ions in the mouth may cause an undesirable taste. A need exits to develop a better way to orally deliver clindamycin

### SUMMARY OF THE INVENTION

This invention relates to a clindamycin salt complex with water insoluble strongly acidic non-spherical resin particles, wherein predominantly all of the resin particles are less than 75 microns in diameter and at least one non-ionic excipient to form a clindamycin-resin complex, wherein the clindamycin-resin complex provides for immediate release of the clindamycin in a target environment. In one embodiment, the inventive subject matter is an oral liquid pharmaceutical formulation made of a complex of clindamycin hydrochloride, a fractionated resin and excipients to stabilize the complex. The pharmaceutical formulation remains stable in deionized water, until the dosage form reaches an ion-rich environment (stomach or intestine), there which the active ingredient clindamycin hydrochloride is released.

The inventive subject matter includes: a liquid pharmaceutical formulation made of: a complex of clindamycin hydrochloride, a water insoluble strongly acidic resin sodium polystyrene sulfonate having non-spherical particles, wherein predominantly all of the resin particles are less than 75 microns in diameter and at least one non-ionic excipient. This oral liquid pharmaceutical formulation provides a surprising immediate release of clindamycin in the target environment and thus can enhance the palatability of the drug and provide good bioavailability as compared to commercialized oral clindamycin palmitate solution.

The inventive subject matter also includes: a pharmaceutical oral suspension having no more than one percent of clindamycin released from the complex in 5 mL of water upon the addition of 1 mL of simulated saliva. An oral suspension is a pharmaceutical formulation where the drug or other components are insoluble in water. The pharmaceutical suspension is made of clindamycin-resin particles wherein the resin is an insoluble strongly acidic resin of sodium polystyrene sulfonate having non-spherical particles, wherein predominantly all of the resin particles are less than 75 microns in diameter and at least one non-ionic excipient.

The inventive subject matter also includes: a process for making a pharmaceutical formulation. The process includes the steps of: mixing clindamycin hydrochloride and an insoluble strongly acidic resin of sodium polystyrene sulfonate having non-spherical particles, wherein predominantly all of the resin particles are less than 75 microns in diameter in an aqueous medium with at least one non-ionic excipient to form a stable complex. The specific irregular shaped non-spherical particles of sodium polystyrene sulfonate (named AMBERLITE IRP 69 (DOW Chemical Company, Midland Mich.)) are obtained by grinding the spherical shaped AMBERLITE IRP 69F (DOW Chemical Company, Midland Mich.) into smaller size particles, wherein predominantly all of the resin particles are less than 75 microns in diameter (fractionated resin).

### BRIEF DESCRIPTION OF SEVERAL VIEWS OF THE INVENTION

The invention description refers to the accompanying drawings:

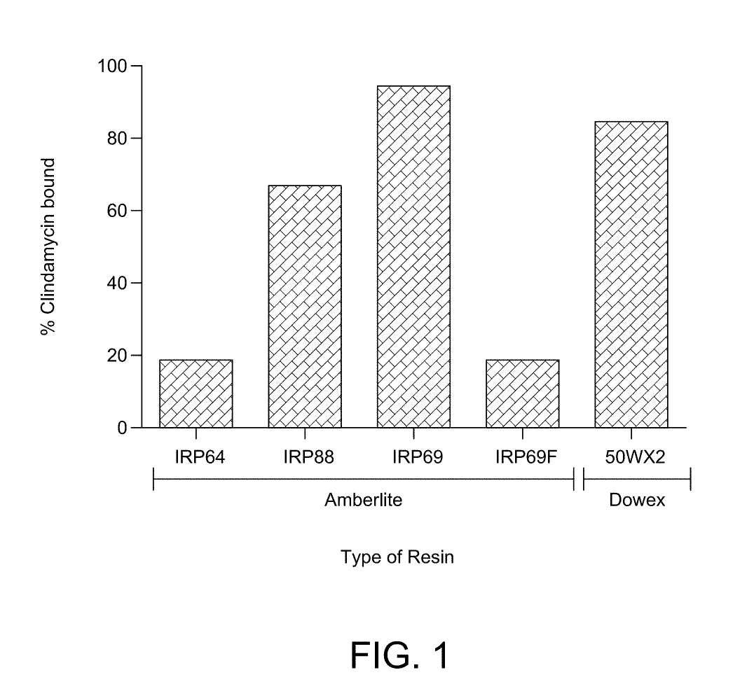

FIG. 1 shows the percentage of clindamycin (75 mg dose of clindamycin HCl) bound to different ion exchange resins in 5 mL of deionized water.

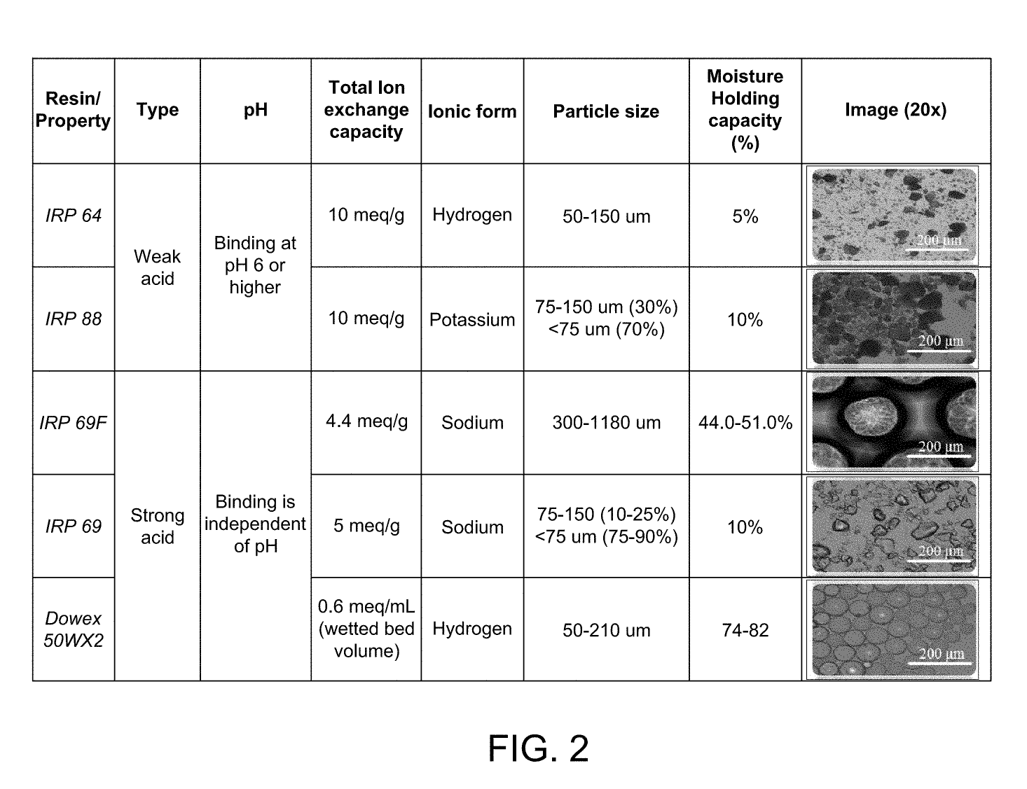

FIG. 2 shows a list of cation exchange resins (IERs) and their physicochemical properties

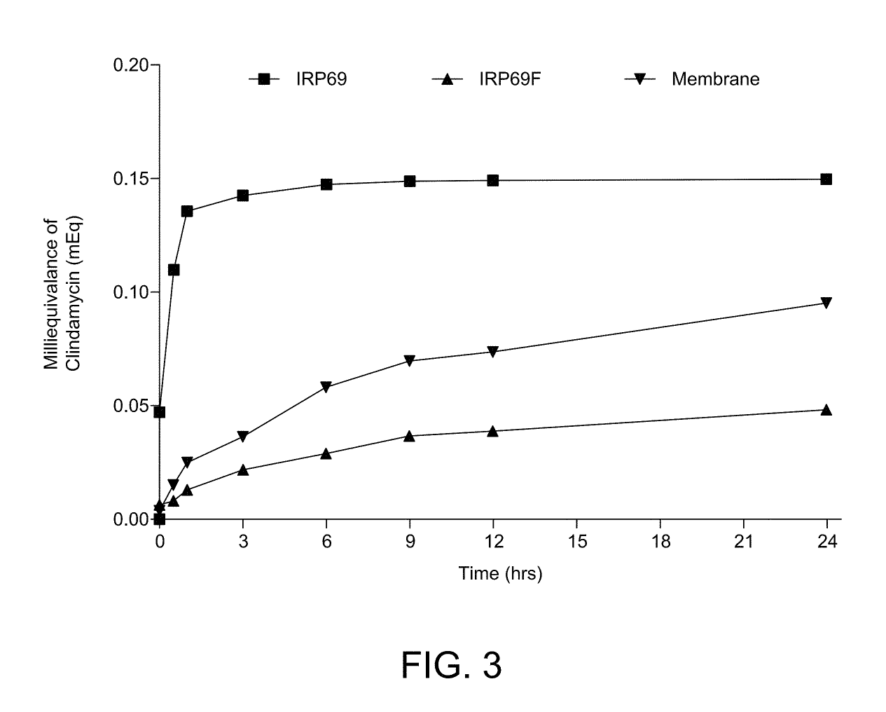

FIG. 3 shows the binding of clindamycin HCl in milliequivalence to an equivalent amount of sodium polystyrene sulfonate in a fractionated form with non-spherical particles (AMBERLITE IRP 69 (DOW Chemical Company, Midland Mich.)), a spherical form with particle size of 300-1180 microns (AMBERLITE IRP 69F (DOW Chemical Company, Midland Mich.)) and a membrane planar form (CMI-4000s Membranes International Inc, Ringwood, N.J.).

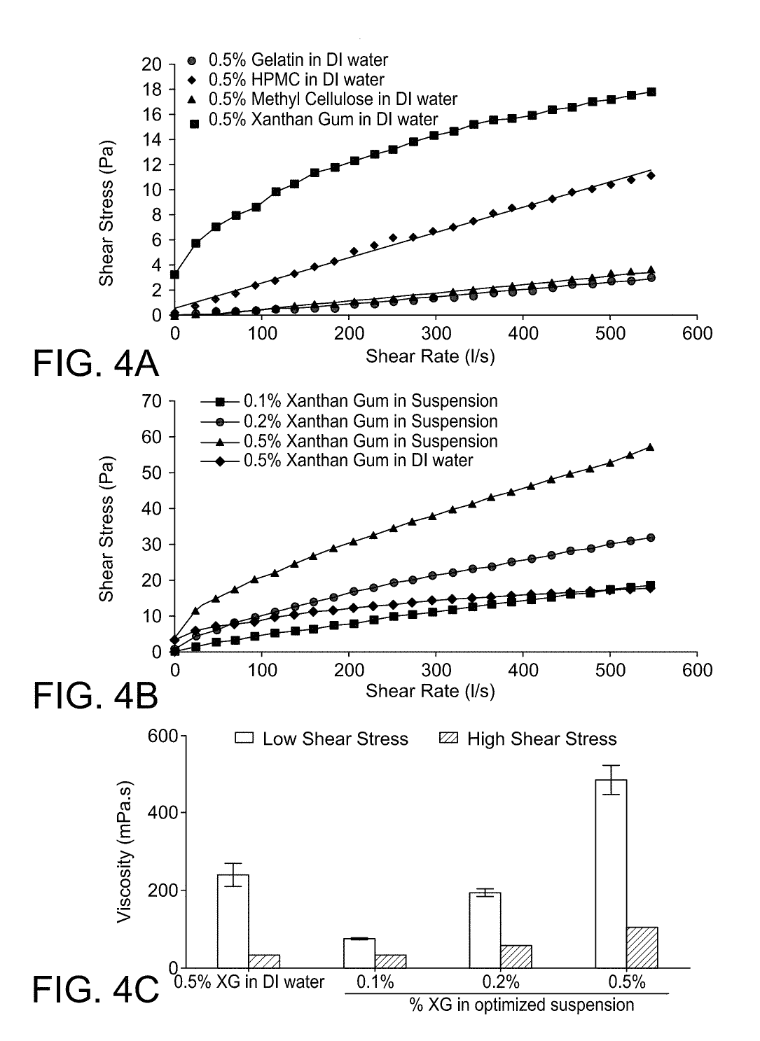

FIGS. 4A-4C shows a (A) shear stress vs shear rate of several suspending agents in deionized water, (B) Shear stress vs shear rate of different concentrations of xanthan gum in suspension formulation and deionized water (C) Viscosity values of different concentrations of xanthan gum in suspension formulation at low and high shear stress.

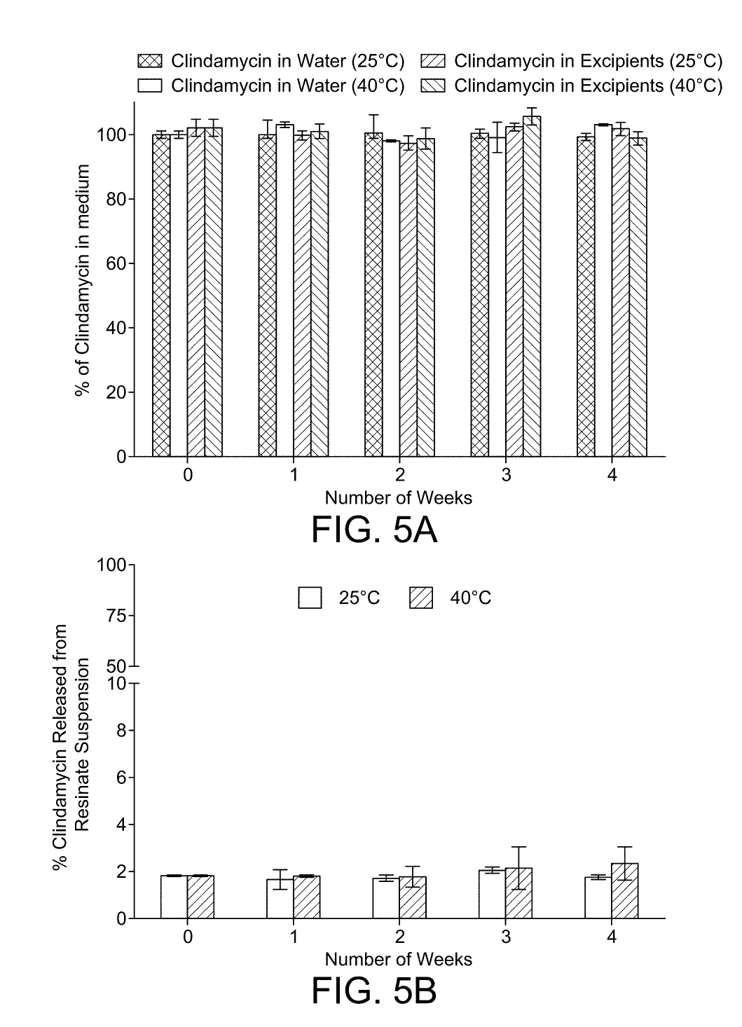

FIGS. 5A-5B shows a (A) Stability profile of 75 mg clindamycin HCl in deionized water and the effect of the excipients used in the optimized suspension on the chemical stability of clindamycin after storage for 1 month at 25° C. and 40° C. (B) The percentage of clindamycin released from resinate into the suspension medium after storage for 1 month at 25° C. and 40° C.

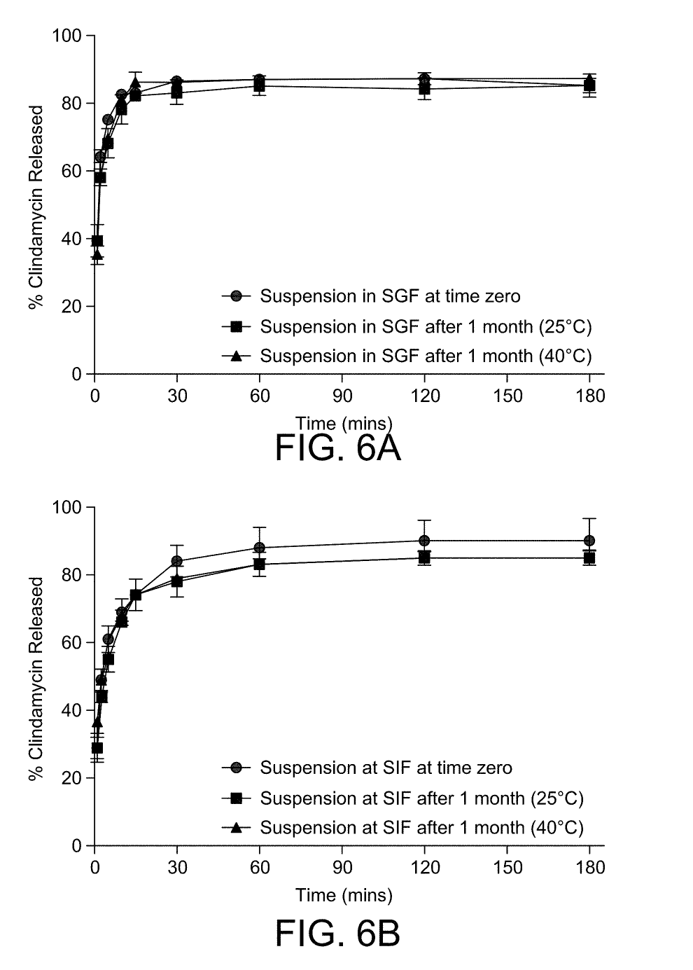

FIGS. 6A-6B shows a A) release profiles of Clindamycin from optimized suspensions at time zero and after 1 month at 25° C. and 40° C. in A) simulated gastric fluid SGF (37° C., 900 ml, 100 rpm) and B) simulated intestinal fluid SIF (37° C., 900 ml, and 100).

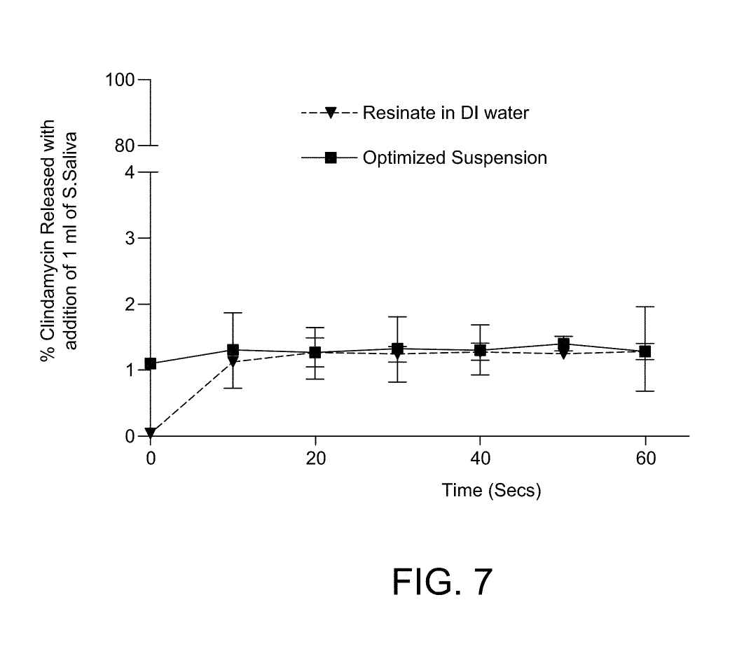

FIG. 7 Clindamycin release during 1 minute from 5 ml of optimized suspension and plain resinate in 5 ml DI water after the addition of 1 ml simulated saliva in 37° C. at 60 rpm.

### DETAILED DESCRIPTION OF SEVERAL EMBODIMENTS

This invention relates to a clindamycin salt complexed with water insoluble strongly acidic non-spherical resin particles, wherein predominantly all of the resin particles are less than 75 microns in diameter. The term predominantly all means that between 75-90% of the particles are less than 75 microns. The clindamycin salt complex includes at least one non-ionic excipient to form clindamycin-resin complex, wherein the clindamycin-resin complex provides for immediate release of the clindamycin in a target environment

In one exemplary embodiment, the inventive subject matter includes an oral liquid pharmaceutical formulation made of a complex of clindamycin hydrochloride, a fractionated resin of sodium polystyrene sulfonate and a sufficient amount of non-ionic excipients to stabilize the complex. The pharmaceutical formulation remains stable in deionized water until the dosage form reaches an ion-rich target environment (stomach or intestine), there which the active ingredient clindamycin hydrochloride is released. In one exemplary embodiment, the unit dose of clindamycin hydrochloride is 75 mg. A unit dose is a certain amount of the drug that shows therapeutic efficacy and minimum side effects.

In the present invention, immediate release and taste masking of clindamycin is achieved by complexation with fractionated AMBERLITE IRP 69 (DOW Chemical Company, Midland Mich.) and with the addition of other excipients. Clindamycin release from the fractioned AMBERLITE IRP 69 (DOW Chemical Company, Midland Mich.) has an immediate release profile and not sustained release as most drug binding to ion exchange resins result in sustained release for 12 hours or more. Plus, taste evaluation of the suspension in adult volunteers concluded that taste masking was successful in eliminating clindamycin bitterness. The example shows that clindamycin in the presence of excipients and in the complex form with the resin in suspension, has good stability over the period of one month.

Now referring to FIG. 1 clindamycin-resin complexes (resinates) were prepared by a single batch process. In this example, several types of resins were evaluated for their selective binding to clindamycin: AMBERLITE IRP 64, IRP 88, IRP 69F, IRP 69 (DOW Chemical Company, Midland Mich.) and DOWEX 50WX2 (DOW Chemical Company, Midland Mich.). A 200 mg aliquot of each resin was weighed and placed in scintillation vials that contained 75 mg of clindamycin hydrochloride/5 ml deionized water. The contents of vials were then mixed and placed in a shaker (New Brunswick Scientific, Enfield, Conn.) at 25° C. with 300 rpm for three hours. At the end of the example (3 hours), the contents of the vials were filtered, and the filtrates were assayed for clindamycin using the HPLC method described below.

The clindamycin resinate was washed three times with deionized water during filtration to remove any free drug that might be loosely attached to the resin. Finally, the resinate complex was recovered from the filter paper and was dried overnight in a desiccator and stored in a tight glass vial. High performance liquid chromatography analytical method to estimate the amount of clindamycin bound to each resin. The amount of Clindamycin in filtrate was assessed using the HPLC. The amounts of bound clindamycin were calculated by subtracting the amounts of detected clindamycin in the medium from the original clindamycin added.

Since clindamycin has an amine group (pKa 7.6), it binds to the negatively charged ionic moiety in the resin. AMBERLITE IRP 64 and 88 (DOW Chemical Company, Midland Mich.) are considered weak acids and have carboxylic acid functional groups, AMBERLITE IRP69 (DOW Chemical Company, Midland Mich.), AMBERLITE IRP 69 F (DOW Chemical Company, Midland Mich.) and DOWEX (DOW Chemical Company, Midland Mich.) are classified as strong acids as they contain sulfonic acid functional groups. A STRONG ACID has a pH in the range of 1-2 and a pKa of 3 or less.

Based on the preliminary equilibrium experiments, the time required to reach binding equilibrium was three hours. 200 mg of the resins was used to bind 75 mg of anhydrous clindamycin HCl (MW 461.45). Based on the data in FIG. 1, AMBERLITE IRP 69 (DOW Chemical Company, Midland Mich.) was used to form the resinate complex and for further suspension formulation development.

Now referring to FIG. 2 the physical characteristics of the resins are described. AMBERLITE IRP 64 (DOW Chemical Company, Midland Mich.) has irregular shape particles with size ranging from 50-150 μm. AMBERLITE IRP 88 (DOW Chemical Company, Midland Mich.) has similar sizes and shapes to AMBERLITE IRP 64 (DOW Chemical Company, Midland Mich.) with particle size ranging from 75-150 μm. AMBERLITE IRP 69F (DOW Chemical Company, Midland Mich.) is spherical with particle size ranging from 300-1180 μm. AMBERLITE IRP 69 (DOW Chemical Company, Midland Mich.) is a fractionated resin (obtained from grinding IRP 69F) has shown irregular shape particles with particle size, wherein predominantly all of the resin particles are less than 75 microns in diameter (between 75-90 percent are less than 75 microns and between 10-25 percent are between 75-150 microns).

DOWEX 50WX2 (DOW Chemical Company, Midland Mich.) is spherical in shape and has particle size ranging from 50-210 μm. Light microscope: The shape and the surface morphology of the resins tested in this example were examined using EVOS microscope (Life Technologies, Grand Island, N.Y.). The dry powder was placed on a glass slide and a slipping cover was placed over it. Images were captured at a magnification of 20×.

Now referring to FIG. 3 clindamycin binding to fractioned resin AMBERLITE IRP69 (DOW Chemical Company, Midland Mich.) indicates a surprisingly immediate release profile and not sustained release as most drug binding to ion exchange resins result in sustained release for 12 hours or more. Immediate release means: 80% of the drug is released within 15-30 minutes in a target environment. FIG. 3 shows the clindamycin binding to AMBERLITE IRP69, IR69F (DOW Chemical Company, Midland Mich.) and CMI-7000 (Membrane Membranes International Inc, Ringwood, N.J.). These three resins have been chosen based on their common structure that includes styrene divinylbenzene copolymer and the ionic sodium sulfonate functional group but are represented in different shapes. Normally, AMBERLITE IRP 69 (DOW Chemical Company, Midland Mich.) is supplied as dry fine powder (45-150 um) which is obtained after grinding of AMBERLITE IRP 69F (DOW Chemical Company, Midland Mich.). AMBERLITE IRP 69F (DOW Chemical Company, Midland Mich.) comes in the form of larger beads (0.3-1.2 mm) and the Membrane is formed as planar sheets. AMBERLITE IRP 69 (DOW Chemical Company, Midland Mich.) is obtained by grinding of AMBERLITE IRP 69F. Equal equivalence of Ionic functional group from AMBERLITE IRP 69 (DOW Chemical Company, Midland Mich.) (200 mg), AMBERLITE IRP 69F (DOW Chemical Company, Midland Mich.) (213 mg) and membrane (625 mg) were weighed and added to 5 ml of DI that contains 75 mg clindamycin HCL. The vials were then placed on a shaker for 3 hours at 300 rpm. At the end of the experiment, resins were filtered, and clear samples were taken for analysis of unbound clindamycin. The analysis of amount of clindamycin bound was performed using HPLC. Data clearly shows the swift binding of clindamycin to AMBERLITE IRP 69 (DOW Chemical Company, Midland Mich.) as compared to AMBERLITE IRP69F (DOW Chemical Company, Midland Mich.) and CMI-7000S (Membranes International Inc, Ringwood, N.J.). This is surprising, as equivalent amounts of sulfonic acids are available for binding. The slow binding of clindamycin to AMBERLITE IRP 69F (DOW Chemical Company, Midland Mich.) as compared to the membrane and AMBERLITE IRP 69 (DOW Chemical Company, Midland Mich.) is indicative of osmotically controlled diffusion and consequently can lead to sustained release behavior in the gastro-intestinal tract.

The present subject matter includes excipients that are compatible with clindamycin resinate and which do not initiate clindamycin release from the resinate complex. Four classes of excipients are included in this subject matter: (1) suspending and viscosity enhancing agents, (2) sweetening agents, (3) surfactants and (4) flavors. Suspending and viscosity inducing agents reduce the sedimentation rate of the resinate particles and minimize the interaction between any clindamycin molecules and the taste buds. Suitable suspending and viscosity inducing agents include: xanthan gum, hydroxymethyl cellulose, methyl cellulose, gelatin and carbomer. Xanthan gum is a natural anionic polysaccharide while hydroxymethyl cellulose and methyl cellulose are non-ionic semi-natural polysaccharide. Gelatin is a heterogeneous mixture of single or multi-stranded polypeptides. Carbomer is an anionic polymer and is defined as a gelling agent that transforms into gel by adding a “neutralizer” or a pH adjusting chemical. The viscosity enhancing agent makes up from between 0.1-2%, preferably 0.5 percent of the total suspension.

In an exemplary embodiment, xanthan gum was selected as the suspending and viscosity inducing agent.

Sweetening agents are used in liquid formulations for their sweetening efficiency. The sweetening agents that showed minimum release <1% of clindamycin from the resinate complex include: sucrose, MAGNASWEET (Mafco, Camden N.J.), maltodextrin, glycerol, and sucralose. Sweetening agents range from about 2-50 percent of the formulation.

The formulation also included surfactants. A surfactant facilitates the homogenous distribution of resinate particles in the medium and prevents aggregation. Additionally, the surfactants of this invention are selected to be non-ionized to avoid any premature release of clindamycin from the resin in the suspension formulation.

In one exemplary embodiment, the non-ionic surfactants are selected from: CREMOPHOR RH 40 (BASF Mannheim, Germany), TWEEN 80 (Sigma Aldrich, Milwaukee, Wis.), (2-[2-[3,4-bis(2-hydroxyethoxy)oxolan-2-yl]-2-(2-hydroxyethoxy)ethoxy]ethyl octadec-9-enoate; 2-{2-[3,4-BIS(2-HYDROXYETHOXY)OXOLAN-2-YL]-2-(2-HYDROXYETHOXY)ETHOXY}ETHYL OCTADEC-9-ENOATE), POLOXAMER 188 (Sigma Aldrich, Milwaukee, Wis.) and POLOXAMER 407 (Sigma Aldrich, Milwaukee, Wis.). In one exemplary embodiment, CREMOPHOR RH 40 (BASF Mannheim, Germany) was selected as the surfactant of choice. The surfactants range from 5 to 20 percent, but preferably 15 percent of the ingredient.

Aqueous-based flavoring agents, in an exemplary embodiment include: maple, grape, strawberry and raspberry. HPLC data showed minimum clindamycin release with maple and grape (˜1%) as compared to strawberry and raspberry (see TABLE 1). The raspberry and strawberry flavors recorded average release of 1.88% and 5.59% after seven days, respectively. This suggests the presence of ionic moieties in these flavors which triggered the release. Based on the results above, maple and grape flavors were selected for an exemplary formulation. The flavoring agents range from 5 to 20 percent, but preferably 10 percent of the ingredient.

TABLE 1
The influence of a single exciplent added on clindamycin released
from resinate in 5 ml of DI water (mean SD).

| Inactive | % of | % of clindamycin Release (75 mg Clindamycin HCl) | | | |
|---|---|---|---|---|---|
| Ingredient | ingredient | Day 1 | Day 3 | Day 5 | Day 7 |
| Viscosity Enhancing Agents | | | | | |
| Xanthan Gum | 0.5 | 0.68 ± 0.003 | 0.68 ± 0.015 | 0.70 ± 0.08 | 0.82 ± 0.005 |
| Hydroxypropyl Methyl Cellulose | 0.5 | 0.14 ± 0.002 | 0.23 ± 0.0007 | 0.22 ± 0.006 | 0.19 ± 0.0016 |
| Methyl Cellulose | 0.5 | 0.07 ± 0.011 | 0.17 ± 0.0017 | 0.18 ± 0.0008 | 0.13 ± 0.0128 |
| Cabomer | 0.5 | N/A | N/A | N/A | N/A |
| Gelatin | 0.5 | 0.32 ± 0.003 | 0.35 ± 0.001 | 0.31 ± 0.003 | 0.50 ± 0.0016 |
| Sweetening Agents | | | | | |
| Sucrose | 50 | 0.19 ± 0.0232 | 0.27 ± 0.001 | 0.27 ± 0.006 | 0.25 ± 0.0007 |
| Glycerol | 50 | 0.05 ± 0.0036 | 0.12 ± 0.002 | 0.13 ± 0.008 | 0.11 ± 0.005 |
| Maltodextrin | 50 | 2.87 ± 0.0036 | 2.60 ± 0.005 | 2.83 ± 0.0141 | 3.76 ± 0.0131 |
| Sucralose | 5 | 0.06 ± 0.0063 | 0.14 ± 0.004 | 0.15 ± 0.0181 | 0.28 ± 0.006 |
| Stevia | 2 | 0.72 ± 0.012 | 0.76 ± 0.026 | 0.62 ± 0.058 | 0.71 ± 0.0345 |
| MAGNASWEET | 2 | 4.48 ± 0.005 | 4.26 ± 0.009 | 4.14 ± 0.005 | 5.43 ± 0.003 |
| Surfactants | | | | | |
| TWEEN 80 | 15 | 0.30 ± 0.003 | 0.46 ± 0.004 | 0.57 ± 0.006 | 0.85 ± 0.033 |
| POLOXAMER407 | 15 | 0.63 ± 0.024 | 0.78 ± 0.004 | 0.78 ± 0.003 | 0.93 ± 0.005 |
| CREMPHOR RH 40 | 15 | 0.59 ± 0.024 | 0.66 ± 0.003 | 0.67 ± 0.003 | 0.71 ± 0.002 |
| Flavoring Agents | | | | | |
| Maple | 10 | 0.42 ± 0.011 | 0.55 ± 0.011 | 0.67 ± 0.15 | 0.5 ± 0.072 |
| Grape | 10 | 0.93 ± 0.012 | 1.17 ± 0.023 | 0.93 ± 0.12 | 1.27 ± 0.005 |
| Strawberry | 10 | 0.79 ± 0.006 | 1.20 ± 0.082 | 1.63 ± 0.008 | 1.88 ± 0.347 |
| Raspberry | 10 | 2.92 ± 0.100 | 3.76 ± 0.002 | 3.68 ± 0.040 | 5.59 ± 0.475 |

In vivo evaluation of the palatability of formulations was evaluated. The criteria and the number of volunteers included in such studies play an important factor in achieving precise evaluation and more accurate representation for outcome. Many taste evaluation studies have been performed using different numbers of volunteers. However, most studies ranged from 6-20 volunteers with selections from either sexes and within certain age groups between 20-35 years old. The criteria for choosing the volunteers is being healthy and of limited age group which is an important factor as taste is one of the senses that is highly affected by age.

The following examples are illustrative only and are not intended to be a limitation on the present invention. Examples: Materials: Several ion exchange resins: AMBERLITE IRP64, (DOW Chemical Company, Midland Mich.); AMBERLITE IRP69 (DOW Chemical Company, Midland Mich.), AMBERLITE IRP69F (DOW Chemical Company, Midland Mich.), AMBERLITE IRP88 (DOW Chemical Company, Midland Mich.) and DOWEX 50WX2) (DOW Chemical Company, Midland Mich.) and Hydroxypropyl methylcellulose (Methocel E50) HPMC were obtained from Dow Chemical Company (Midland, Mich.). Clindamycin hydrochloride was purchased from Accela ChemBio Co., Ltd. (Shanghai, China). Glycerin, TWEEN 80 (Sigma Aldrich, Milwaukee, Wis.) and HPLC grade acetonitrile solvent were purchased from Fisher Chemicals (Pittsburgh, Pa.). Maltodextrin (Dextrose equivalent 4-7), sorbitol F solution, methyl cellulose (MC), sodium chloride, potassium chloride, sodium bicarbonate, monopotassium phosphate, hydrochloric acid and sodium phosphate dibasic dodecahydrate were obtained from Sigma Aldrich (St. Louis, Mo.). Xanthan gum, gelatin, sucrose, stevia and POLOXAMER 407 (Sigma Aldrich, Milwaukee, Wis.) were purchased from Gallipot (St. Paul, Minn.). Maple syrup, grape flavor, strawberry flavor, raspberry flavor was obtained from LorAnn Oils (Lansing, Mich.). Sucralose was purchased from Alfa Aesar (Ward Hill, Mass.). CREMOPHOR RH 40 (BASF Mannheim, Germany) was obtained from BASF (Mannheim, Germany). MAGNASWEET (Mafco worldwide Corp. (Camden, N.J.), was obtained from Mafco worldwide Corp. (Camden, N.J.). CARBAPOL (Lubrizol Corporation Wickliffe, Ohio) was purchased from Lubrizol Corporation (Wickliffe, Ohio). Millipore deionized water was used in this example. Selection of excipients: The influence of each excipient on the release of clindamycin from AMBERLITE IRP 69 (DOW Chemical Company, Midland Mich.) resinate was evaluated.

It is preferred for a drug-resinate suspension is to show no release of clindamycin from resinate complex when excipients are added. For this purpose, several viscosity enhancing agents (xanthan gum, hydroxypropyl methyl cellulose, methyl cellulose, carbomer, and gelatin), several sweetening agents (sucrose, glycerol, maltodextrin, sucralose, stevia, and MAGNASWEET (Mafco, Camden N.J.), several surfactants (TWEEN 80 (Sigma Aldrich, Milwaukee, Wis., POLOXAMER 407 (Sigma Aldrich, Milwaukee, Wis.), and CREMOPHOR (BASF Mannheim, Germany) and flavoring agents (maple, grape, strawberry, raspberry) were evaluated. The amounts used were equivalent to the percentages listed in TABLE 1 and were added to 275 mg of Clindamycin resinate in vials which contained 5 ml DI water. Each vial of clindamycin resinate with single excipient was shaken vigorously and then placed in 25° C. incubator. Clindamycin release was detected over 1, 3, 5 and 7 days' period. At each sampling time, vials were vortexed for 10 seconds and 100 μl samples were taken and filtered using 0.22 μm filters. The filtrates were diluted with DI water and quantified for clindamycin using the aforementioned HPLC method.

Based on the preliminary excipients screening, one excipient from each subclass was selected for the optimized suspension formulation. A 5 ml of the suspension was prepared according to the following approach: 275 mg of resinate were weighted in a vial, CREMOPHOR RH40), (BASF Mannheim, Germany) was added and mixed until a homogenous paste was obtained. The other ingredients were then added separately in a separate vial according to the percentages stated in TABLE 2. The content of the second vial was added to the paste in the first vial then all components were adequately mixed and the final suspension was finally formed.

TABLE 2
The Formulation of the Optimized Suspension

| Ingredient | Percentage (w/v %) |
|---|---|
| Clindamycin | 5.5 (275 mg) |
| resinate | |
| Glycerin | 30 |
| Sucralose | 3 |
| Maple flavor | 7 |
| Grape flavor | 10 |
| Cremophore RH 40 | 15 |
| Xanthan gum | 0.2 |
| D.I. water | q.s to 5 ml |

Characterization of clindamycin released in SGF and SIF. The clindamycin release profiles from the optimized suspension formulation and from resinate powder were examined using USP dissolution apparatus II, paddle (Distek Evolution 6100, North Brunswick, N.J.). Five milliliters of the suspension formulation (equivalent to 275 mg resinate) or 275 mg resinate (dry powder) were added to 900 ml of either simulated gastric or simulated intestinal fluids at 37±0.5° C. and 100 rpm. Small samples of 0.5 mL were withdrawn at predetermined time points (0.5, 2.5, 5, 10, 15, 30, 60, 120 and 180 minutes) and filtered using 0.22 um filters and assayed for clindamycin released using HPLC method described previously. The formulation of SGF is 0.2% (w/v) Sodium Chloride in 0.7% (v/v) Hydrochloric Acid (11). The formulation of SIF is potassium monophosphate (6.805 g/L) and sodium hydroxide (0.896 g/L)

Stability and compatibility example of the optimized complex. Stability evaluation of free clindamycin HCl in DI water, free clindamycin HCl with excipients and clindamycin resinate in the suspension formulation was conducted over a one-month period at 25° C. and 40° C. In details, 75 mg clindamycin HCL dissolved in 5 ml DI water, 75 mg clindamycin HCL dissolved in 5 ml of DI water with excipients (no resin) and clindamycin resinate in suspension formulation (5 ml) were incorporated in 10 ml sealed glass vials and were kept in incubators at 25° C. and 40° C. using EXCELLA E24 incubator (New Brunswick Scientific, Enfield, Conn.) and NAPCO 5400 incubator (Thermo Fisher Scientific, West Palm Beach, Fla.), respectively. Samples were collected once a week and then filtered and assayed for clindamycin content using HPLC method. As part of stability example, the release profile of clindamycin from the optimized suspension formulation which were kept at 25° C. and 40° C. after 0 and 1 month was also evaluated in SGF and SIF as described in the in vitro release section.

Taste Evaluation example: Determination of the bitterness threshold for clindamycin in Table 3. The determination of the bitter taste threshold of clindamycin was carried out in 4 adult volunteers according to the following procedures: In brief, series of different concentrations of clindamycin (5, 10, 15, 20, 25 μg/ml) were prepared in DI water. Four volunteers were asked to rinse their mouths with 10 ml DI water then taste the clindamycin solutions (5 mL) by swirling it thoroughly into their mouths for 30 seconds starting with the most dilute solution (5 μg/ml). After 30 seconds, the residual solution in the mouth was washed away and a one-minute waiting period was requested to account for any delayed sensitivity. A ten-minute gap was assigned between the first and the next highest concentration of testing solutions. All the volunteers recorded their results depending upon the concentration by which they started to sense the bitter taste. The bitterness threshold of clindamycin was selected as the minimum average concentration of clindamycin that triggered the bitter taste.

TABLE 3
Bitterness threshold of clindamycin in aqueous solution

| | Concentration (μg/ml) | | | | |
|---|---|---|---|---|---|
| Volunteers | 5 | 10 | 15 | 20 | 25 |
| 1 | | X | | | |
| 2 | | X | | | |
| 3 | | | X | | |
| 4 | | | X | | |

Full taste masking evaluation was carried out for the purpose of evaluating the taste masking efficiency of IER in the optimized suspension formulation. Five milliliters of suspension formulation containing 275 mg clindamycin resinate were tested against reference solution of 75 mg clindamycin HCL in 30% Sucrose solution (reference solution). This example was carried out according to a clinical protocol approved by the IRB at the University of Tennessee. Twelve healthy adult volunteers between the ages of 25-35 representing both genders (9 males, 3 females) were chosen. All volunteers were asked to rinse their mouths with water for 30 seconds before taking any preparation. Next, each volunteer was asked to retain (with constant swirling) either 5 ml of suspension or 5 ml or reference solution in their mouths for 15 seconds. After expectoration, the volunteers were asked to record the bitterness score through a numerical scale from 1=most disliked to 10=highly liked. A gap of 1 hour was kept as a wash out period between testing of suspension and reference solution to avoid any interferences.

TABLE 1 shows the influence of several excipients on the release of clindamycin from AMBERLITE IRP 69 (DOW Chemical Company, Midland Mich.) resinate complex in 5 mL of DI water. It was noted that all suspending agents at 0.5% have shown very low release of clindamycin from resinate over 7 days' period (about less than 1% of the clindamycin dose). The sweetening agents also showed similar behavior to the suspending agents except maltodextrin and MAGNASWEET (Mafco, Camden N.J.). Both showed clindamycin release between 2.6-5.4% of dose. The surfactants on the other hand had similar release to viscosity agents (less than 1%). Finally, flavoring agents had mixed results where maple had release less than 1%, grape and strawberry had a release ranging between 0.7-2%. Raspberry flavor had much higher release ranging between 2.92-5.6% of the dose.

The optimum formulation of clindamycin suspension. Based on the clindamycin release results in TABLE 1, the excipients which showed the minimum release of clindamycin were selected for the suspension formulation. TABLE 2 shows the percentages of excipients selected to form the optimum suspension formulation. In this regard, 30% of glycerin, 3% of sucralose, 7% of maple, 10% of grape, 15% of CREMOPHOR RH40 (BASF Mannheim, Germany) and 0.2% of xanthan gum were selected for this exemplary purpose.

Rheological studies: The four polymers selected (gelatin, HPMC, methyl cellulose, xanthan gum) were studied at a concentration of 0.5% in DI water. This was done to evaluate their rheological behavior before inclusion into the suspension. FIG. 4A shows the rheological profile of each polymer as shear stress increases gradually against the shear rate. As seen in the FIG. 4A all polymers except xanthan gum showed nearly straight line which resembles the typical rheological behavior of Newtonian systems. This might be attributed to the low percentage (0.5%) for each polymer used. This means that higher concentrations are needed for the shear thinning effect to become dominant. However, xanthan gum showed shear thinning pattern at 0.5% as it is apparent from the curve in FIG. 4A.

FIG. 4B shows the shear stress versus shear rate for different concentrations of xanthan gum at 0.1, 0.2 and 0.5% in suspension as compared to 0.5% in distilled water. All profiles of xanthan gum showed shear thinning behavior. In comparison, the profile of 0.5% xanthan gum in distilled water lied between the 0.1-0.2% of xanthan gum in the suspension. FIG. 4C presents the values for the viscosities at low and high shear stresses. At low shear stress (shelf state), xanthan gum in distilled water at 0.5% showed an average value of 250 mPa·s which is very close to 0.2% xanthan gum in the optimized suspension. Viscosity values for xanthan gum at 0.1% and 0.5% in the suspension were 80 mPa·s and 500 mPa·s respectively.

At high shear stress which resembles the state at which the patient shakes the suspension vigorously to attain a good pourability, the viscosity values were reduced significantly as shown in the FIG. 4C. This clearly exhibits the shear thinning properties of xanthan gum in the suspension. Also, the viscosity of 0.5% xanthan gum in water (33 mPa·s) was very close to the value in 0.2% xanthan gum in the suspension (59 mPa·s). Thus, based on the data in FIGS. 4A, B, and C, it was concluded that 0.2% of xanthan gum would be sufficient for the preparation of the suspension.

In vitro release of clindamycin in SGF and SIF. The objective of the in vitro dissolution testing is to predict the in vivo performance of pharmaceutical preparations. Therefore, SGF and SIF were used in this example to mimic the environment in the gastrointestinal environment.

Stability studies of clindamycin in resinate and suspension. Clindamycin is stable in aqueous solutions and in most buffers at room temperature. To confirm the stability of clindamycin in DI water, 75 mg clindamycin HCl (equivalent to the commercial dose) were dissolved in 5 ml water and were tested over one month at 25 and 40° C. Separately, clindamycin in solution was also evaluated in the presence of other excipients used in the suspension (excluding the resin AMBERLITE IRP 69 (DOW Chemical Company, Midland Mich.)) over a one month period to observe any incompatibility or degradation which can be initiated in the presence of these excipients.

As seen in FIG. 5A, clindamycin shows good stability profile with no degradation over the one-month period of example at 25° C. and 40° C. Clindamycin concentration remained close to the initial amount after one month. Likewise, no degradation of clindamycin was apparent under the influence of any of the excipients added. Moreover, no extra peaks or shifts in the peaks were observed in HPLC chromatogram during the analysis of the samples at all-time points.

As seen in FIG. 5B, the stability of clindamycin bound to resinate was evaluated over a one-month period. The purpose of this was to assess whether there is any effect coming from the excipients on the release of clindamycin bound to the resinate in the suspension over the one-month period. It was evident that at 25° C. and 40° C., there were no significant changes in the percentage of clindamycin released from the resinate at initial storage (˜1.8%) as compared to any time point during the month (2%). This means that the excipients have no effect on the bound clindamycin during storage for at least 1 month.

FIG. 6 shows the stability of clindamycin bound to resinate in the suspension. This example was carried out by comparing the release profiles of clindamycin in optimized suspension at time zero and after 1 month at 25 and 40° C. using SGF and SIF as dissolution media. No significant differences were noticed in the release profiles of clindamycin from all suspensions at either at 25° C. or 40° C. at SGF or SIF. In either media, about 90% of clindamycin was released from suspension after one month at 25 and 40° C. This is a clear indication of the stability of clindamycin in the suspension and a strong indication that the release is immediate release and not sustained release.

FIG. 7 shows the characterization of clindamycin released in simulated saliva. The release of clindamycin from suspension formulation and from resinate powder in simulated saliva solution (SS) was determined. This example was utilized to simulate the extent of clindamycin released in the mouth. Five milliliters of optimized suspension formulation or 275 mg of resinate in 5 ml DI water were added to 1 ml of heated SS at 37° C. to mimic the conditions into the mouth. The reason for selecting 1 ml of saliva was based on several reports which indicated that the volume of the human saliva to be about 1 ml. Samples were withdrawn at 10 second intervals during mixing at 60 rpm using precision general-purpose water bath (Thermo Fisher Scientific, Werest Palm Beach, Fla.) and Clindamycin contents were assayed after filtration using the HPLC method. The simulated saliva SS consists of potassium chloride (0.149 mg/ml), sodium chloride (0.117 mg/ml) and sodium bicarbonate (2.1 mg/ml). Each example was repeated in triplicate and the average percentage of clindamycin released was calculated at each time interval.

### Taste Evaluation Example

Determination of bitterness threshold of clindamycin. When drug molecules enter the oral cavity, taste buds are activated upon the exposure. However, in order to initiate a response, a certain amount of the drug has to bind to the taste buds. This process initiates bitter or other types of taste (sweet, sour, etc.). In this example, a bitterness recognition threshold is to be reached when the drug concentration reaches a certain value at which the bitterness taste is felt. As the concentration of the drug increases, the intensity of bitterness subsequently increases until it reaches a plateau, upon which a higher drug concentration will have no additional influence on bitterness intensity. Moreover, the determination of the bitterness threshold for a drug is essential for evaluating its bitterness intensity. It has been reported that drugs have different bitterness thresholds which come as a manifestation of the affinity of each drug to the taste buds receptors. In this example, clindamycin bitterness threshold was determined using four adult volunteers. Two of the volunteers felt the bitterness of clindamycin at 10 μg/ml while the other two volunteers felt the bitterness at 15 μg/ml (see TABLE 4). The differences in recorded values are linked to the interpersonal variability in taste sensitivity.

Therefore, 12.5 μg/ml was considered as the mean bitterness threshold for clindamycin taste. This value is equivalent to approximately 0.1% of the clindamycin dose used in this example. Consequently, this suggested that a very low exposure of clindamycin to the taste buds will trigger a sense of bitterness.

TABLE 4
Scores of Taste Evaluation Example:

| | Scores | | | | | | | | | |
|---|---|---|---|---|---|---|---|---|---|---|
| | 1 | 2 | 3 | 4 | 5 | 6 | 7 | 8 | 9 | 10 |
| Preparation/Score | Number of volunteers corresponding to each score | | | | | | | | | |
| Optimized | | | | | | | | 2 | 6 | 4 |
| Suspension | | | | | | | | | | |
| Clindamycin in 30% | 5 | 5 | 2 | | | | | | | |
| sucrose | | | | | | | | | | |

Moreover, in this example here, 12 volunteers were carefully chosen and the taste evaluation was performed based on the volunteers providing a grading on a 1 to 10 scale for the optimized suspension against clindamycin HCl in 30% sucrose as a reference solution. Data was expressed as the number of volunteers who reported the same grading number. The volunteers rated the optimized suspension at a score ranging between 8 and 10 which indicated that the suspension formulation was highly liked (see TABLE 4). In contrast, reference solution of clindamycin in 30% sucrose scored a very low score 1-3 which indicated a high level of dislike by the volunteers. The difference in taste between the optimized suspension and aqueous solution is highly in favor of the formulation and shows that the bitterness of clindamycin was successfully masked.

While the invention has been described with reference to preferred and example embodiments, it will be understood by those skilled in the art that a variety of modifications, additions and deletions are within the scope of the invention, as defined by the following claims.

1. A pharmaceutical composition comprising:
a clindamycin salt complexed with strongly acidic non-spherical resin particles, wherein predominantly all of the resin particles are less than 75 microns in diameter and at least one non-ionic excipient to form a clindamycin-resin complex, wherein in the clindamycin-resin complex provide for immediate release of the clindamycin in a target environment.

2. The pharmaceutical composition of claim 1, wherein the at least one non-ionic excipient is a viscosity enhancing agent selected from the group consisting of: xanthan gum, hydroxy methyl cellulose, methyl cellulose, gelatin and carbomer or combinations thereof.

3. The pharmaceutical composition of claim 1, further comprising: a sweetening agent selected from the group consisting of: sucrose, glycerol, sucralose, maltodextrin, MAGNASWEET (Mafco, Camden N.J.), and stevia or combinations thereof.

4. The pharmaceutical composition of claim 1, wherein the at least one non-ionic excipient is a surfactant selected from the group consisting of: TWEEN 80 (Sigma Aldrich, Milwaukee, Wis.), POLOXAMER 407 (Sigma Aldrich, Milwaukee, Wis.), POLOXAMER 188 (Sigma Aldrich, Milwaukee, Wis.) and CREMPHOR RH 40 (Sigma Aldrich, Milwaukee, Wis.) or combinations thereof.

5. The pharmaceutical composition of claim 1, wherein the at least one non-ionic excipient is a flavoring agent ranging from 5 to 20 percent of the pharmaceutical composition selected from the group consisting of: maple, grape, strawberry and raspberry or combinations thereof.

6. The pharmaceutical composition of claim 1, wherein the resin is a fractionated sodium polystyrene sulfonate resin, wherein between 75-90 percent of the resin particles are less than 75 microns and between 10-25 percent are between 75-150 microns.

7. A pharmaceutical composition of claim 1, wherein the clindamycin salt is clindamycin hydrochloride, which is in the form of a unit dose, in which the amount of the unit dose of clindamycin hydrochloride is 75 mg.

8. A pharmaceutical suspension having no more than 1 percent release of clindamycin in 5 mL of deionized water comprising:
a clindamycin resinate, wherein the clindamycin resinate is formed of a resin made of an insoluble strongly acidic resin having non-spherical particles, wherein predominantly all of the resin particles are less than 75 microns in diameter;
at least one non-ionic excipient; and
a sufficient amount of deionized water to form the pharmaceutical suspension, wherein the suspension is formulated for immediate release of clindamycin in a target environment.

9. The pharmaceutical suspension of claim 8, wherein the resin is a fractionated sodium polystyrene sulfonate resin, wherein between 75-90 percent of the resin particles are less than 75 microns and between 10-25 percent are between 75-150 microns.

10. The pharmaceutical suspension of claim 8, wherein the non-ionic excipient is a viscosity enhancing agent comprising about 0.5 percent of the pharmaceutical suspension, wherein the viscosity enhancing agent is selected from the group consisting of: xanthan gum, hydroxy methyl cellulose, methyl cellulose, gelatin and carbomer or combinations thereof.

11. The pharmaceutical suspension of claim 8 wherein the non-ionic excipient is a sweetening agent ranging from about 2-50 percent of the suspension, wherein the sweetening agent is selected from the group consisting of: sucrose, glycerol, sucralose, maltodextrin, MAGNASWEET (Mafco, Camden N.J.), and stevia or combinations thereof

12. The pharmaceutical suspension of claim 8, wherein the non-ionic excipient is a surfactant ranging from 5 to 20 percent of the pharmaceutical suspension, said surfactant selected from the group consisting of: TWEEN 80 (Sigma Aldrich, Milwaukee, Wis.), POLOXAMER 407 (Sigma Aldrich, Milwaukee, Wis.) POLOXAMER 188 (Sigma Aldrich, Milwaukee, Wis.) and CREMPHOR RH 40 (Sigma Aldrich, Milwaukee, Wis.) or combinations thereof.

13. The pharmaceutical suspension of claim 8, wherein the non-ionic excipient is selected from the group consisting of: glycerin; sucrose; a flavoring agent; CREMOPHOR RH 40 (BASF Mannheim, Germany) and xantham gum or combinations thereof.

14. The pharmaceutical suspension of claim 8, wherein the non-ionic excipient is a flavoring agent ranging from 5 to 20 percent of the pharmaceutical suspension, said flavoring agent is selected from the group consisting of: maple and grape flavors, or a combination thereof.

15. A process for making an oral liquid pharmaceutical formulation of clindamycin hydrochloride and a fractionated resin which comprises the steps of: mixing clindamycin hydrochloride and a fractionated resin, the fractionated resin comprised of: an insoluble strongly acidic resin having non-spherical particles, wherein predominantly all of the resin particles are less than 75 microns in diameter, together in an aqueous medium to form a stable clindamycin resinate complex.

16. The process according to claim 15 in which the molar ratio of the fractionated resin to clindamycin hydrochloride is 1 meq:0.15 meq.

17. The process of claim 15 further comprising the step of adding a buffering agent to adjust the pH to 6.5

18. The process according to claim 15 further comprising the step of adding at least one non-ionic excipient.

19. The process of claim 15 further comprising the step of adding a sufficient amount of xanthan gum to suspend the stable clindamycin resinate complex.

20. The process of claim 19 wherein in the amount of xanthan gum is 0.5 percent of the formulation.
